(19) United States
(12) Patent Application Publication
(10) Pub. No.: US 2011/0097369 A1
(43) Pub. Date: Apr. 28, 2011

(54) Particulate Detergent Additive

(76) Inventors: Matthias Sunder, Duesseldorf (DE); Mario Sturm, Leverkusen (DE); Tobias Segler, Duesseldorf (DE); Noelle Wrubbel, Duesseldorf (DE)

(21) Appl. No.: 12/983,344

(22) Filed: Jan. 3, 2011

Related U.S. Application Data
(63) Continuation of application No. PCT/EP2009/056906, filed on Jun. 5, 2009.

(30) Foreign Application Priority Data
Jul. 3, 2008 (DE) 10 2008 031 212.6

(51) Int. Cl. C11D 17/00; A61K 8/891; A61Q 19/00; A61K 8/11; C11D 7/60

(52) U.S. Cl. 424/401; 424/600; 510/349; 510/513

(57) ABSTRACT

Laundry-detergent and cleaning-product additives in particle form, comprising a water-soluble or water-dispersible carrier and active ingredient microcapsules. These particles allow a user to obtain particular advantages with respect to fragrancing and care of the articles treated in conventional laundering and cleaning operations, such as, in particular, in automatic laundering of fabrics.

### CROSS REFERENCE TO RELATED APPLICATIONS

The present application is a continuation of International Patent Application No. PCT/EP2009/056906 filed 5 Jun. 2009, which claims priority to German Patent Application No. 10 2008 031 212.6 filed 3 Jul. 2008, both of which are incorporated herein by reference.

The present invention relates to particles suitable for use in laundry-detergent products, cleaning products and care products. The particles have a water-soluble or water-dispersible carrier and active ingredient microcapsules. Furthermore, the present invention relates to a method for producing such particles, as well as detergents, cleaning agents or care agents containing such particles. Finally, it also relates to the use of such products in textile laundry and/or textile treatment.

In laundering textiles, a user typically not only pursues the goal of removing soil from laundry for hygienic and visual purposes, but also desires an added value that goes beyond merely cleaning the textile. This added value includes, for example, textiles smelling good after laundering or yielding a softer feel when laundered. Consumers have a particularly great interest in pleasant smelling laundry.

For this reason, and to mask any inherent odor of the textile detergent, most commercially available textile detergents contain fragrances. However, often only a relatively weak scent remains on the laundry when traditional detergents are used, particularly when a dryer is used.

Against this background, the present invention provides a means that enables a consumer to acquire an added value as part of the traditional machine treatment of textiles, so that this value goes beyond the mere cleaning of textiles.

This is achieved by the subject matter of the invention, namely by a particle suitable for use in laundry-detergent products, cleaning products or care products, comprising a water-soluble or water-dispersible carrier as well as active ingredient microcapsules.

Microcapsules as such are known. The diameters of usable microcapsules can range from a few nanometers to millimeters. Solid and/or liquid active ingredients are enclosed in the microcapsules according to the invention. High-molecular compounds are usually used as materials for the capsules such as protein compounds (e.g., gelatin, albumin, casein and others) and cellulose derivatives (e.g., methylcellulose, ethylcellulose, cellulose acetate, cellulose nitrate, carboxymethylcellulose and others), as well as synthetic polymers (e.g., polyamides, polyethylene glycols, polyurethanes, epoxy resins and others). In this regard, further details will be given below. The general principle of microencapsulation is known in particular as monoencapsulation of liquid or solid phases by sheathing with film-forming polymers (e.g., the aforementioned polymers). The film formers are deposited on the material to be enclosed after emulsification and coacervation or interfacial polymerization. Active ingredient microcapsules (e.g., microcapsules containing fragrances) are widely available commercially.

Particles according to the invention can be used in the main wash cycle of an automatic washing or cleaning method, particularly as an extra additive added in addition to a normal detergent or cleaning agent or as an integral component of a detergent or cleaning agent. The particles may be added, for example, together with the detergent or cleaning agent to the washing drum or to the rinse compartment of a washing machine.

Active ingredients contained in the microcapsules can contribute toward achieving the added value, which goes beyond the simple cleaning of textiles. According to the invention, this added value may be manifested in an improved textile fragrancing, an improved textile care and/or even in achieving cosmetic skin-care effects, depending on the choice of active ingredients implemented.

Particles according to the invention also comprise a water-soluble or water-dispersible carrier as a component in addition to the microcapsules. Water-soluble or water-dispersible carriers comprising material(s) chosen from inorganic alkali metal salts, organic alkali metal salts, inorganic alkaline earth metal salts, organic alkaline earth metal salts, organic acids, carbohydrates, silicates, urea or mixtures thereof is a preferred embodiment of the invention. Such carrier materials are not only inexpensive, but usually also dissolve very well in water. Furthermore, these materials are odor-neutral.

Suitable materials include inorganic alkali metal salts such as sodium chloride, potassium chloride, sodium sulfate, sodium carbonate, potassium sulfate, potassium carbonate, sodium bicarbonate, potassium bicarbonate or mixtures thereof, organic alkali metal salts such as sodium acetate, potassium acetate, sodium citrate, sodium tartrate or potassium sodium tartrate, inorganic alkaline earth metal salts such as calcium chloride, magnesium sulfate or magnesium chloride, organic alkaline earth metal salts such as calcium lactate, carbohydrates, organic acids, such as citric acid or tartaric acid, silicates such as water glass, sodium silicate or potassium silicate, urea and mixtures thereof.

Especially preferred water-soluble or water-dispersible carriers however, comprise carbohydrates. Thus, if the water-soluble or water-dispersible carriers include a carbohydrate chosen from dextrose, fructose, galactose, isoglucose, glucose, sucrose, raffinose or mixtures thereof, this is also a preferred embodiment of the invention. It is particularly advantageous if the water-soluble or water-dispersible carrier used is based on at least about 80 wt. % carbohydrates, preferably at least about 90 wt. %, in particular at least about 95 wt. %, or even completely carbohydrates.

Useful carbohydrates include rock sugar or sugar crystals. Using crystalline sugar yields particles that are especially appealing esthetically and met with greater consumer acceptance. According to a preferred embodiment of the invention, the particles include a carrier present in the form of crystals.

The water-soluble or water-dispersible carrier can also contain mixtures of the aforementioned materials (e.g., mixtures of salts such as sodium citrate) and carbohydrates.

When using a water-soluble or water-dispersible carrier consisting of carbohydrates and/or at least predominantly carbohydrates, corrosion in the washing machine is avoided; however, this could occur when using inorganic chloride salts as the water-soluble or water-dispersible carrier.

In another preferred embodiment, the proportion of water-soluble or water-dispersible carrier amounts to 50 to 99 wt %, preferably 75 to 95 wt %, based on total particles.

According to another preferred embodiment of the invention, microcapsules according to the invention also contain a preferably liquid active ingredient suitable for laundry, cleaning, care and/or finishing purposes, such as—
(a) fragrances,
(b) textile-care ingredients such as preferably silicone oils, cationic polymers, and/or
(c) skin-care ingredients, preferably vitamin E, natural oils, aloe vera extract, green tea extract, D-panthenol, plankton extract, vitamin C, urea and/or glycine.

The microcapsules can also readily contain solids (e.g., in the form of dispersions), for example, extremely fine hydrophobic silica, finely distributed in a perfume oil.

Some statements about fragrances, textile-care ingredients and skin-care ingredients are made below. It should be noted that all these substances may be present in and/or on particles according to the invention, as well as both inside and outside the microcapsules.

If skin-care ingredients (preferably as active ingredients in microcapsules) are used, they preferably manifest their effect indirectly via the treated textile, which transfers the skin-care ingredient to the skin on coming in contract with the latter, so that the skin can then draw a cosmetic benefit from it.

The skin-care ingredient is preferably hydrophobic, maybe liquid or solid. Examples of skin-care ingredients that may be used include—
a) waxes such as carnauba, spermaceti, beeswax, lanolin derivatives thereof and mixtures thereof;
b) plant extracts, for example, vegetable oils such as avocado oil, olive oil, palm oil, palm kernel oil, rapeseed oil, linseed oil, soy oil, peanut oil, coriander oil, castor oil, poppy seed oil, cocoa oil, coconut oil, pumpkin seed oil, wheat germ oil, sesame oil, sunflower oil, almond oil, macadamia nut oil, apricot kernel oil, hazel nut oil, jojoba oil, canola oil, chamomile or aloe vera as well as mixtures thereof;
c) higher fatty acids such as lauric acid, myristic acid, palmitic acid, stearic acid, behenic acid, oleic acid, linoleic acid, linolenic acid, isostearic acid or polyunsaturated fatty acids;
d) higher fatty alcohols such as lauryl alcohol, cetyl alcohol, stearyl alcohol, oleyl alcohol, behenyl alcohol or 2-hexadecanol;
e) esters such as cetyl octanoate, lauryl lactate, myristyl lactate, cetyl lactate, isopropyl myristate, myristyl myristate, isopropyl palmitate, isopropyl adipate, butyl stearate, decyl oleate, cholesterol isostearate, glycerol monostearate, glycerol distearate, glycerol tristearate, alkyl lactate, alkyl citrate or alkyl tartrate;
f) hydrocarbons such as paraffins, mineral oils, squalane or squalene;
g) lipids;
h) vitamins such as A, C and/or E and/or vitamin alkyl esters;
i) phospholipids;
j) sunscreen agents such as octylmethoxyl cinnamate and butylmethoxybenzoylmethane;
k) silicone oils, such as linear or cyclic polydimethylsiloxanes, amino-, alkyl-, alkylaryl- or aryl-substituted silicone oils, and
l) mixtures thereof.

Most preferred, however, are fragrances, particularly in combination with textile-care ingredients (e.g., silicone oil) and/or in combination with skin-care ingredients (e.g., with almond oil, etc.).

In laundering textiles, the consumer expects not only visually satisfactory cleanliness, but also the absence of any unpleasant odors on the cleaned textiles. The persistence of fragrances which originate from detergents and ensure a pleasant odor is perceived as particularly pleasant and reinforces the cleanliness impression. For washed laundry, consumers want a scent that is noticeable not only in the product itself and immediately after washing, but also one that can be perceived on the treated object for several days.

However, the amount of perfume absorbed on textiles (e.g., from an aqueous solution from the washing or rinsing process) is often too small to ensure a perceptible scent impression over a longer period of time. Since fragrances are especially cost-intensive ingredients of detergents and cleaning agents, they tend to be used only in small amounts. Loss of these ingredients (e.g., in a washing machine) is equally unsatisfactory for manufacturers and consumers of such agents.

It has now been found that by using particles according to the invention, when they contain fragrance, an especially advantageous scent impression (increased appeal/higher intensity/better persistence) can be achieved in washing and/or cleaning surfaces, particularly textiles, especially when the particles used contain water-insoluble fragrance microcapsules.

Individual fragrance compounds (e.g., the synthetic products of the type of esters, ethers, aldehydes, ketones, alcohols and hydrocarbons) may be used as fragrances and/or perfume oils and/or scents (these terms are used synonymously here). However, mixtures of different fragrances are preferably used together creating an appealing scent note. Such perfume oils may also contain natural fragrance mixtures such as those accessible from plant sources.

It is particularly advantageous to use perfume oils generally associated with certain impressions. Perfume oil advantageously awakens associations with impression such as “cleanliness” and “freshness,” which are associated with the use of detergents in general. Perfume oil may advantageously support the impression of “care.” For example, it is advantageous to incorporate a plurality of fragrances which support the “care” perception into the microcapsules and to incorporate a plurality of fragrances which awaken associations with impressions such as “cleanliness” and “freshness” into particles outside of the microcapsules or vice versa.

Within the scope of this invention, fragrances which can be used advantageously to impart and/or accompany the impression of “cleanliness” and “freshness” include bergamot oil, tangerine oil, dimethyl anthranilate, aldehyde C 11(en), dihydromyrcenol, 4-tert-butylcyclohexyl acetate, allylamyl glycolate, tetrahydrolinalool, 6-methyl-gamma-ionone, isobornyl acetate, cyclovertal, ethyl linalool, aldehyde C 12, dynascone 10, limonene, orange oil, isobornyl acetate, eucalyptus oil (globulus), calone, cyclovertal, ethyl-2-methyl butyrate, tetrahydrolinalool, aldehyde C 10, styrolyl acetate, otbca, water fruit base, citronitrile, undecavertol, styrolyl acetate, tonalide and/or dihydromethyl jasmonate, in particular, however, dihydromyrcenol and/or 4-tert-butylcyclohexyl acetate. Consequently, preferred perfume oils may include at least one of the aforementioned fragrances.

Preferred fragrances which can be used to enhance and/or accompany the impression of a “care effect” within the scope of this invention include aldehyde C 14, decalactone gamma, cyclamen aldehyde, lilial, troenan, canthoxal, citronellol, geraniol, musk, phenylethyl alcohol, dihydrofloriffone, dmbca, phenirate, phenylethyl isobutyrate, rose oxide, jasmelia, hexyl cinnamaldehyde (alpha), jonone beta, ylang, cyclohexyl salicylates, hexenyl salicylates (cis-3), sandelice, santobar, bacdanol, guaiac wood oil, iso E super, timerol (forte), norlimbanol, ambroxan, cinnamyl alcohol, cyclopentadecanolide, nirvanol, javanol, aldehyde C 11, habanolide, maltol, benzyl acetone, coumarin, benzyl salicylates, melonal, galbanum oil, ethyl vanillin, koavone, ptbca 25 cis, hedione, lilial, dihydrofloriffone, isoraldein, methyl palmitate, methyl oleate and/or methyl myristate. Consequently, preferred perfume oils may include at least one of the aforementioned fragrances.

According to another preferred embodiment, the product according to the invention contains at least one fragrance, preferably two, three or more fragrances from the list of galaxolide, dihydromyrcenol, 4-tert-butylcyclohexyl acetate, gamma-isomethylionone, tetrahydrolinalool, hexyl cinnamaldehyde, lilial, linalool, amyl cinnamaldehyde, 6-methyl-gamma-ionone, methyl oleate, neryl acetate, 15-pentadecalactone, phenoxyethyl isobutyrate, phenylethyl methanolate, α-pinene, β-pinene, rose oxide, sabinene, anethol, benzoic acid 2-hydroxypentyl ester, diphenyl ether, benzophenone, cyclamen aldehyde, α-damascone, decanal, dicyclopentadiene alcohol allylcyclohexyl propionate, isobornyl acetate, bornyl acetate, dihydromethyl jasmonate, eucalyptol, n-dodecanol, ethyl palmitate, geraniol acetate, hexyl acetate, n-hexyl salicylates, α-ionone, methyl palmitate, 2-naphthyl methyl ketone, isopropyl myristate, rose phenone, widdrene, styrallyl acetate, thujopsene, dimethylbenzylcarbinyl butyrate, limonene, dimethylbenzylcarbinyl acetate, citronellol, 2-tert-butylcyclohexanol, caryophyllene, ethyl stearate, tonalide, 2,4-hexadienal, methanoazulene, methyl laurate, methyl myristate, 2-methyl undecanal, myrcene, nonanal, nopyl acetate, 15-pentadecalactone, beta-phellandrene, 3-phenyl-2-methylpropene, rose acetate, traseolide and/or α-terpineol.

Use of scent precursors is also very advantageous, preferably when they are contained in the (preferably water-insoluble) microcapsule. A scent precursor is a compound which releases a desired odor and/or scent molecule by, for example, breaking a chemical bond by hydrolysis. To form a scent precursor, typically a desired scent raw material is chemically bonded to a carrier, preferably a slightly volatile or moderately volatile carrier. This combination leads to a less volatile and more strongly hydrophobic scent precursor with improved addition onto substances. The scent is then released by breaking the bond between the scent raw material and the carrier, for example, by a change in pH (e.g., due to transpiration while being worn), atmospheric humidity, heat and/or sunlight during storage or drying on the laundry line.

Scent raw materials useful in scent precursors are typically saturated or unsaturated volatile compounds containing an alcohol, an aldehyde and/or a ketone group. Scent raw materials that are useful here may include any pleasant-smelling substances or mixture of substances.

Especially advantageous scent precursors that may be used according to the present invention conform to the formula—

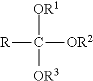

where R is hydrogen, linear C₁-C₈ alkyl, branched C₃-C₂₀ alkyl, cyclic C₃-C₂₀ alkyl, branched cyclic C₆-C₂₀ alkyl, linear C₆-C₂₀ alkenyl, branched C₆-C₂₀ alkenyl, cyclic C₆-C₂₀ alkenyl, branched cyclic C₆-C₂₀ alkenyl, substituted or unsubstituted C₆-C₂₀ aryl and mixtures thereof; R′, R² and R³ independently are linear, branched or substituted C₁-C₂₀ alkyl; linear, branched or substituted C₂-C₂₀ alkenyl; substituted or unsubstituted cyclic C₃-C₂₀ alkyl; substituted or unsubstituted C₆-C₂₀ aryl, substituted or unsubstituted C₂-C₄₀ alkyleneoxy; substituted or unsubstituted C₃-C₄₀ alkyleneoxyalkyl; substituted or unsubstituted C₆-C₄₀ alkylenearyl; substituted or unsubstituted C₆-C₃₂ aryloxy; substituted or unsubstituted C₆-C₄₀ alkyleneoxyaryl; C₆-C₄₀ oxyalkylenearyl and mixtures thereof. The use of such substances, particularly in microcapsules (preferably water-insoluble) corresponds to a preferred embodiment of the invention.

It is a preferred embodiment when the scent precursor that may be used according to the invention releases compounds conforming to the formula—

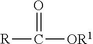

where R is hydrogen, methyl, ethyl, phenyl and mixtures thereof; R¹ is chosen from 4-(1-methylethyl)cyclohexenemethyl, 2,4-dimethyl-3-cyclohexen-1-ylmethyl, 2,4-dimethylcyclohex-1-ylmethyl, 2,4,6-trimethyl-3-cyclohexen-1-ylmethyl, 2-phenylethyl, 1-(4-isopropylcyclohexyl)ethyl, 2,2-dimethyl-3-(3-methylphenyl)propan-1-yl, 3-phenyl-2-propen-1-yl, 2-methyl-4-(2,2,3-trimethyl-3-cyclopenten-1-yl)-2-buten-1-yl, 3-methyl-5-phenylpentan-1-yl, 3-methyl-5-(2,2,3-trimethyl-3-cyclopenten-1-yl)-4-penten-2-yl, 2-methyl-4-phenylpentan-1-yl, cis-3-hexen-1-yl, 3,7-dimethyl-6-octen-1-yl, 3,7-dimethyl-2,6-octadien-1-yl, 7-methoxy-3,7-dimethyloctan-2-yl, 6,8-dimethylnonan-2-yl, cis-6-nonen-1-yl, 2,6-nonadien-1-yl, 4-methyl-3-decen-5-yl, benzyl, 2-methoxy-4-(1-propenyl)phenyl, 2-methoxy-4-(2-propenyl)phenyl and mixtures thereof. The use of such substances, in particular in the microcapsules (preferably water-insoluble) corresponds to a preferred embodiment of the invention.

Additional especially advantageous scent precursors that may be used according to the invention include acetals or ketals, preferably conforming to the formula—

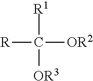

where R is linear C₁-C₂₀ alkyl, branched C₃-C₂₀ alkyl, cyclic C₆-C₂₀ alkyl, branched cyclic C₆-C₂₀ alkyl, linear C₂-C₂₀ alkenyl, branched C₃-C₂₀ alkenyl, cyclic C₆-C₂₀ alkenyl, branched cyclic C₆-C₂₀ alkenyl, substituted or unsubstituted C₆-C₂₀ aryl and mixtures thereof; R¹ is hydrogen or R; R² and R³, independently of one another, are each chosen from linear C₁-C₂₀ alkyl, branched C₃-C₂₀ alkyl, cyclic C₃-C₂₀ alkyl, branched cyclic C₆-C₂₀ alkyl, linear C₆-C₂₀ alkenyl, branched C₆-C₂₀ alkenyl, cyclic C₆-C₂₀ alkenyl, branched cyclic C₆-C₂₀ alkenyl, C₆-C₂₀ aryl, substituted C₇-C₂₀ aryl and mixtures thereof. Use of such substances, particularly in the microcapsules (preferably water-insoluble) corresponds to a preferred embodiment of the invention.

Additional especially advantageous scent precursors that may be used according to the invention conform to the formula—

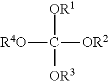

where R¹, R², R³ and R⁴ independently of one another are linear, branched or substituted C₁-C₂₀ alkyl; linear, branched or substituted C₂-C₂₀ alkenyl, substituted or unsubstituted cyclic C₅-C₂₀ alkyl; substituted or unsubstituted C₆-C₂₀ aryl, substituted or unsubstituted C₂-C₄₀ alkyleneoxy; substituted or unsubstituted C₃-C₄₀ alkyleneoxyalkyl; substituted or unsubstituted C₆-C₄₀ alkylenearyl; substituted or unsubstituted C₆-C₃₂ aryloxy; substituted or unsubstituted C₆-C₄₀ alkyleneoxyaryl; C₆-C₄₀ oxyalkylenearyl; and mixtures thereof. Use of such substances, particularly in the (preferably water-insoluble) microcapsules corresponds to a preferred embodiment of the invention.

It is especially preferred that fragrance substances used include silicic acid ester mixtures containing silicic acid esters of the formulae—

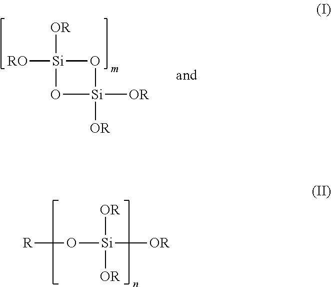

where all the R radicals, independently of one another, are chosen from H, linear or branched, saturated or unsaturated, substituted or unsubstituted C₁-C₆ hydrocarbon radicals and the scent alcohol radicals and/or biocide alcohol radicals, m assumes values in the range from 1 to 20, and n assumes values in the range from 2 to 100. Preferably at least one of the R radicals in formula I and one in formula II are a scent alcohol radical and/or a biocidal alcohol radical. The silicic acid ester mixtures preferably constitute at least 2 wt % of the total amount of fragrance, where wt % is based on all the fragrances in the total particle. Silicic acid ester mixtures are used in the (preferably water-insoluble) microcapsules in particular.

Especially suitable scent precursors include the reaction products of compounds comprising at least one primary and/or secondary amino group, for example, an amino-functional polymer, particularly an amino-functional silicone, and a scent ingredient chosen from ketone, aldehyde and mixtures thereof. Use of such substances, particularly in the microcapsules (preferably water-insoluble), corresponds to a preferred embodiment of the invention.

Perfume oils contained in the particle, particularly in the (preferably water-insoluble) microcapsules, comprising fragrances having a boiling point of 250° C. or greater and a log P value ≧3.0 represent a preferred embodiment.

Use of such fragrances, particularly in the (preferably water-insoluble) microcapsules allows a further improvement in the scent effect with regard to pleasure, intensity and endurance of the scent impression.

Perfume oils contained in the (preferably water-insoluble) microcapsules containing at least 1, 5 or 10 wt % fragrances (wt % based on perfume oil contained in the microcapsules) with a boiling point of 250° C. or greater and a log P value ≧3.0 represent a preferred embodiment. It has been found that particles according to the invention containing such minimal amounts of fragrances with a boiling point of 250° C. or greater and a log P value ≧3.0 in the (preferably water-insoluble) microcapsules have especially advantageous scent properties. For example, an even longer-lasting scent impression can be achieved in the laundry.

The octanol/water distribution coefficient of a scent ingredient refers to the ratio between its equilibrium concentration in octanol and in water. Since distribution coefficients of scent ingredients often have high values (e.g., 1000 or higher), they are expediently expressed in the form of their logarithm to the base 10; thus, referred to as the so-called log P value.

The log P values of numerous fragrances are documented. For example, the Pomona92 database available from Daylight Chemical Information Systems, Inc. (“Daylight CIS”), Irvine, Calif., contains numerous log P values together with citations from the original literature. However, log P values are most expediently calculated by the C LOG P program, also available from Daylight CIS. This program includes experimental log P values to the extent available in the Pomona92 database. The “calculated log P” (C log P value) is [obtained] by fragment approximation according to Harsch and Leo (see A. Leo, Comprehensive Medicinal Chemistry, C. Harsch, P. G. Sammens, J. B. Taylor and C. A. Ransden, Eds., Vol. 4, p. 295, Pergamon Press (1990)). Fragment approximation is based on the chemical structure of each of the scent constituents and takes into account the numbers and types of atoms and atomic bonding power, as well as the chemical bond. C log P values, the most reliable and most widely used estimates for this physicochemical property, are preferably used within the scope of this invention instead of the experimental log P values when selecting scent constituents useful in the present invention.

Boiling points of numerous fragrances are given, for example, in “Perfume and Flavor Chemicals (Aroma Chemicals),” S. Arctander, published by the author in 1969. Other boiling points may be obtained from, for example, various known chemical handbooks and databases. If a boiling point is given only at a different pressure than standard pressure of 760 mmHg, usually a lower pressure, the boiling point at standard pressure can be approximated using boiling point-pressure nomographs, such as those in The Chemist's Companion, A. J. Gordon and R. A. Ford, John Wiley & Sons Publishers, pp. 30-36 (1972). Where applicable, boiling point values may also be calculated by computer programs based on molecular structure data, such as those described in “Computer-Assisted Prediction of Normal Boiling Points of Pyrans and Pynoles”, D. T. Starton et al., J. Chem. Inf. Comput. Sci., Vol. 32, pp. 306-316 (1992); “Computer-Assisted Prediction of Normal Boiling Points of Furans, Tetrahydrofurans and Thiophenes”, D. T. Starton et al., J. Chem. Inf. Comput. Sci., Vol. 31, pp. 301-310 (1992) and the references cited therein; and “Predicting Physical Properties from Molecular Structure”, R. Murugan et al., Chemtech., pp. 17-23 (June 1994).

Table 1 lists a few fragrances as examples of those meeting the criteria of boiling point of 250° C. or greater and C log P ≧3.

TABLE 1
Examples of Useful Fragrances

| | | |
|---|---|---|
| | Approximate | |
| Scent ingredients | boiling point (° C.) | ClogP |

Boiling point of 250° C. or greater and ClogP ≧ 3.0

| | | |
|---|---|---|
| Allyl cyclohexene propionate | 267 | 3.935 |
| Ambrettolide | 300 | 6.261 |
| Amyl benzoate | 262 | 3.417 |
| Amy cinnamate | 310 | 3.771 |
| Amyl cinnamaldehyde | 285 | 4.324 |
| Amyl cinnamaldehyde dimethylacetal | 300 | 4.033 |
| Isoamyl salicylates | 277 | 4.601 |
| Aurantiol | 450 | 4.216 |
| Benzophenone | 306 | 3.120 |
| Benzyl salicylates | 300 | 4.383 |
| para-tert-Butylcyclohexyl acetate | >250 | 4.019 |
| Isobutylquinoline | 252 | 4.193 |
| beta-Caryophylline | 256 | 6.333 |
| Cardinene | 275 | 7.346 |
| Cedrol | 291 | 4.530 |
| Cedryl acetate | 303 | 5.436 |
| Cedryl formate | >250 | 5.070 |
| Cinnamyl cinnamate | 370 | 5.480 |
| Cyclohexyl salicylate | 304 | 5.265 |
| Cyclamen aldehyde | 270 | 3.680 |
| Dihydroisojasmonate | >300 | 3.009 |
| Diphenylmethane | 262 | 4.059 |
| Diphenyl oxide | 252 | 4.240 |
| Dodecanelactone | 258 | 4.359 |
| iso E super | >250 | 3.455 |
| Ethyl brassylate | 3321 | 4.554 |
| Ethylmethylphenyl glycidate | 260 | 3.165 |
| Ethyl undecylenate | 264 | 4.888 |
| Exaltolide | 280 | 5.346 |
| Galaxolide | >250 | 5.482 |
| Geranyl anthranilate | 312 | 4.216 |
| Geranylphenyl acetate | >250 | 5.233 |
| Hexadecanolide | 294 | 6.805 |
| Hexenyl salicylates | 271 | 4.716 |
| Hexyl cinnamaldehyde | 305 | 5.473 |
| Hexyl salicylate | 290 | 5.260 |
| alpha-Iron | 250 | 3.820 |
| Lilial (p-t-bucinal) | 258 | 3.858 |
| Linalyl benzoate | 263 | 5.233 |
| 2-Methoxynaphthalene | 274 | 3.235 |
| Methyl dihydrojasmone | >300 | 4.843 |
| gamma-n-Methylionene | 252 | 4.309 |
| Musk indanone | >250 | 5.458 |
| Musk ketone | M.P. = 137° C. | 3.014 |
| Musk tibetine | M.P. = 136° C. | 3.831 |
| Myristicine | 276 | 3.200 |
| Oxahexadecanolide 10 | >300 | 4.336 |
| Oxahexadecanolide 11 | M.P. = 35° C. | 4.336 |
| Patchouli alcohol | 285 | 4.530 |
| Phantolide | 288 | 5.977 |
| Phenylethyl benzoate | 300 | 4.058 |
| Phenylethylphenyl acetate | 325 | 3.767 |
| Phenylheptanol | 261 | 3.478 |
| Phenylhexanol | 258 | 3.299 |
| alpha-Santalol | 301 | 3.800 |
| Thibetolide | 280 | 6.246 |
| delta-Undecalactone | 290 | 3.830 |
| gamma-Undecalactone | 297 | 4.140 |
| Vetiveryl acetate | 285 | 4.882 |
| Yara-yara | 274 | 3.235 |

Regardless of whether the microcapsules contain fragrances and/or perfume oil, the particle can also contain fragrances and/or perfume oil outside of the microcapsules. Accordingly, a preferred embodiment of the invention relates to a particle containing perfume oil outside of the microcapsules, wherein the composition of the perfume oil outside of the microcapsules preferably differs from the perfume oil optionally contained inside the microcapsules.

It is especially preferred if perfume oil is contained in and/or on the particles both inside the microcapsules and outside of the microcapsules. These perfume oils may be the same, but it is preferable that these perfume oils differ in order to be able to generate an additional scent impression.

One advantage of particles according to the invention containing fragrance (preferably water-insoluble) microcapsules may be seen in the fact that ordinary perfume-loaded sugar crystals tend to require high perfume concentrations in order to achieve, for example, a desired long-lasting fragrancing effect on textiles treated with them. Use of encapsulated perfume oils (preferably water-insoluble), particularly with long-lasting properties, allows a more effective and thus resource-conserving use of perfume oils.

High perfume content may also lead to technical process difficulties in application of the perfume, particularly with respect to perfume-loaded sugar crystals. Due to the limited absorptivity of the crystals, fragrances can be applied basically only on the surface (e.g., in combination with a coating layer). For example, if a perfume-PEG melt is used for coating the crystals, then the melting point of the PEG is greatly reduced when the perfume content is high, thereby inhibiting solidification of the mixture. Consequently, development of a stable coating layer is problematical. Such problems are addressed by the present invention.

Particles containing an amount of perfume from 0.1 to 30 wt %, preferably 0.3 to 15 wt % and in particular 0.5 to 7 wt %, where wt % is based on the total particle, represent a preferred embodiment of the invention.

Microcapsules containing perfume oil in an amount of from 0.01-20 wt %, preferably 0.05-10 wt %, where wt % is based on the total particle, represent a preferred embodiment of the invention.

Another preferred embodiment of the invention is obtained when the amount of perfume oil not contained in the microcapsules is 0-10 wt %, preferably 0.05-5 wt %, where wt % is based on the total particle.

As previously explained, active ingredients in the microcapsules can also include textile-care ingredients. In this way, it is possible to provide detergents or cleaning agents and/or additives with textile-care properties. Further, in the cleaning of textiles, not only are the textiles washed cleanly, but they are also cared for in such a way that, for example, a pleasantly soft feel is imparted to them.

Textile-care ingredients may be present in the particles according to the invention inside the microcapsules and/or outside of the microcapsules.

Particles according to the invention as a textile-care ingredient may advantageously include textile-softening clays. Since textile-softening clays also have a water-softening effect, lime deposits on the laundry are additionally prevented.

In particular, the softening clay can be applied outside of the microcapsules. If softening clay is to be applied to the particle, then it is possible, for example, to first coat the water-soluble or water-dispersible carrier with the softening clay and then to apply microcapsules and optionally thermoplastic polymer. Alternatively, a mixture of microcapsules, softening clay and optionally thermoplastic polymer may also be applied. Alternatively, textile-softening clay may also be applied by dusting in conclusion, corresponding to an especially preferred embodiment.

Smectite clay is an example of a suitable textile-softening clay. Preferred smectite clays include beidellite clays, hectorite clays, laponite clays, montmorillonite clays, nontronite clays, saponite clays, sauconite clays and mixtures thereof. Montmorillonite clays are preferred softening clays. Bentonites contain mainly montmorillonites and may serve as a preferred source of the softening clay. For example, suitable bentonites are distributed under the brand names Laundrosil® by the company Süd-Chemie or under the brand name Detercal by the company Lavlosa.

The amount of textile-softening clay in particles according to the invention can be from about 0.1 to about 10 wt % and preferably 1 to 5 wt %, for example. According to another embodiment, no textile-softening clay is present in particles according to the invention or only very small amounts (e.g., ≦0.1 wt %). A reasonable upper limit may also be 15 wt %, for example.

A main component which may be used in combination with the fabric softening clay or independently thereof is an organic fatty acid softener. This may also be present in particles according to the invention inside the microcapsules and/or outside of the microcapsules. The organic softener may consist of anionic, cationic or nonionic fat chains (C₁₀-C₂₂, preferably C₁₂-C₁₈). Anionic softeners include fatty acid soaps. Preferred organic softeners are nonionic compounds such as fatty acid esters, ethoxylated fatty acid esters, fatty alcohols and polyol polymers. The organic softener is most preferably a higher fatty acid ester of a pentaerythritol compound, where this expression is used in this description to describe higher acid esters of pentaerythritol, higher fatty acid esters of pentaerythritol oligomers, higher fatty acid esters of low alkylene oxide derivatives of pentaerythritol and higher fatty acid esters of low alkylene oxide derivatives of pentaerythritol oligomers.

Particles according to the invention may be contained as a possible textile-care ingredient, for example, a textile-softening polymer, in particular a polysiloxane and/or a cationic polymer. The textile-softening polymer can be present inside and/or outside of the microcapsules. Suitable cationic polymers include those described in CTFA International Cosmetic Ingredient Dictionary, 4^{th} Ed., J. M. Nikitakis et al., Eds., published by the Cosmetic, Toiletry and Fragrance Association 1991 and summarized under the collective term “polyquaternium”. Cationic polymers have a textile-softening effect and thus a textile-care effect and may additionally make a skin-care contribution. Particles according to the invention can also include other suitable textile-care compounds, preferably fluorescent agents, antiredeposition agents, optical brighteners, graying inhibitors, shrinkage preventers, wrinkle control agents, dye transfer inhibitors, antimicrobial active ingredients, germicides, fungicides, antioxidants, antistatics, ironing aids, UV absorbers, phobicizing agents, and/or impregnation agents.

In addition, particles according to the invention can also include a thermoplastic polymer. Particles including a thermoplastic polymer, preferably in amounts of 0.01-25 wt %, particularly 0.05-10 wt %, represent a preferred embodiment of the invention. Polyethylene glycols (PEG), polyvinyl alcohols, polyacrylates, PVP or polyesters are preferably suitable as the thermoplastic polymer. Especially suitable are polyethylene glycols that are solid at room temperature and have a melting point of approximately 65° C.±20° C., for example, a melting point of approximately 60° C. or, for example, 65° C. or, for example, approximately 55° C.

In addition, a particle according to the invention can also include water-binding substances. Particles according to the invention that include water-binding substances, preferably in amounts of 0-20 wt %, particularly 0.1-10 wt %, where wt % is based on total particles, where the water-binding substance is chosen from zeolite, silica, textile-softening clay, starch and/or derivatives thereof and/or cellulose (derivatives), such as preferably carboxymethylcellulose, represent a preferred embodiment of the invention.

In particular, it is preferable if a particle according to the invention is characterized in that the water-soluble or water-dispersible carrier is coated with a mixture comprising thermoplastic polymer and microcapsules. For example, water-binding substances and water may also be present in the optional coating. For example, in a suitable embodiment, the particle core is formed by the water-soluble or water-dispersible carrier, where the core is covered with thermoplastic polymer and microcapsules.

It is preferable for a particle according to the invention, in particular a particle coated with thermoplastic polymer and microcapsules, to also be dusted with a dusting agent, comprising in particular zeolite, silica, textile-softening clay (e.g., bentonite), starch and/or derivatives thereof and/or cellulose (derivatives) such as preferably carboxymethylcellulose. This corresponds to a preferred embodiment of the invention.

In another possible embodiment of the invention, the particle according to the invention is free of surface-active agents, softeners and builders.

Microcapsules usable according to the invention can be water-soluble and/or water-insoluble microcapsules, but are preferably water-insoluble microcapsules. Water insolubility of the microcapsules has the advantage that a separation of active ingredients persisting beyond the laundry application can be made possible in this way. It is particularly preferable if the water-insoluble microcapsules are rupturable microcapsules, wherein the wall material of the microcapsules comprises polyurethanes, polyolefins, polyamides, polyesters, polysaccharides, epoxy resins, silicone resins and/or polycondensation products of carbonyl compounds and compounds containing NH groups. The term “rupturable microcapsules” refers to microcapsules which, when they adhere to a textile treated therewith, can be opened (i.e., ruptured) by mechanical rubbing or by pressure, so that the ingredients are released only as a result of a mechanical action, for example, when one is drying one's hands with a hand towel on which such microcapsules have been deposited. Preferred microcapsules for use here have average diameters in the range of about 0.05 to about 500 μm, preferably about 5 to about 150 μm, in particular 10 to about 100 μm, for example, about 10 to about 80 μm. The shell of the microcapsules enclosing the core and/or (filled) cavity has an average thickness in the range from about 0.01 to about 50 μm, preferably approximately 0.1 μm to approximately 30 μm, particularly from approximately 0.5 μm to approximately 8 μm. Microcapsules are rupturable in particular when they are within the ranges given above with regard to average diameter and average thickness.

The person skilled in the art will be familiar with procedures for producing microcapsules. Suitable methods of producing microcapsules are described, for example, in U.S. Pat. Nos. 3,870,542, 3,516,941, and 3,415,758, as well as in European Patent Application Publication No. 0 026 914 A1. The latter describes, for example, the production of microcapsules by acid-induced condensation of melamine-formaldehyde precondensates and/or their C₁-C₄ alkyl ethers in water, wherein the hydrophobic material forming the capsule core is dispersed, in the presence of a protective colloid. For example, melamine-urea-formaldehyde microcapsules or melamine-formaldehyde microcapsules or urea-formaldehyde microcapsules may preferably be used. These microcapsules are available from the 3M Corporation or from BASF, for example. Microcapsules that may be used are also described in European Patent Application Publication No. 1 244 768 A2.

In the production of particles, the microcapsules can be processed, for example, directly in the dispersion, as often performed in typical production process. The dispersion may optionally be modified, for example, thickened, and/or the water content of the dispersion may be adjusted, so that it contains 5 to 80 wt %, preferably 40 to 80 wt % microcapsules. The microcapsule dispersion can also be mixed first with water-binding substances. This corresponds to a preferred embodiment of the invention. The slurry may also be modified, for example, by using thickeners or by adjusting the water content. In another aspect, the microcapsules can also be used in dry (powder) form instead of dispersed form.

A preferred particle according to the invention is characterized in that the water-soluble or water-dispersible carrier has a particle size in the range of 0.1 to 30 mm, particularly 0.2 to 7 mm and most preferably 0.5 to 3 mm, for example, in the range of 0.8 to 2.5 mm.

The particle as such may have a particle size in the range of ≧0.1 to 30 mm, preferably ≧0.2 to 10 mm, particularly ≧0.5 to 5 mm, for example, in the range of 0.8 to 3 mm.

To improve the esthetic impression of the particles, they can be dyed with suitable dyes. Preferred dyes, the selection of which does not pose any problems for the person skilled in the art, have high storage stability and are insensitive to light and other ingredients in the detergents or cleaning agents, as well as have a pronounced substantivity with respect to textile fibers, so as not to discolor them.

A particle according to the invention may also contain a pearlizing agent for increasing the gloss. Examples of suitable pearlizing agents include ethylene glycol monostearate and distearate (for example, Cutina® AGS from Cognis) as well as PEG-3 distearate.

The particles of the present invention may preferably have a bulk density in the range of 300 to 900 g/L or 400 to 800 g/L, for example, in the vicinity of 650 g/L.

Another aspect of the present invention is directed towards a method for producing particles as described above, comprising—
(a) producing a mixture of microcapsules and thermoplastic polymer, such as preferably PEG, PVA, polyacrylates, PVP or polyester, in the form of a melt containing microcapsules, and
(b) combining the melt from step (a) with water-soluble or water-dispersible carrier material.

Steps (a) and (b) can be performed in typical mixing equipment.

The microcapsules in step (a) can be added in dry form or as an aqueous slurry.

Mixing the microcapsules in the melt together with water-binding substances in step (a) represents a preferred embodiment of the invention. The slurry may also be modified, for example, by using thickeners or by adjusting the water content.

If the water-soluble or water-dispersible carrier material used in step (b) of the method according to the invention has been premodified by mixing the actual carrier with textile-softening clay in the presence of textile-care or skin-care compounds and/or in the presence of perfume in particular, this is another preferred embodiment of the invention.

If the particle is also dusted with a dusting agent, preferably comprising textile-softening clay, in the method according to step (b) of the invention, this is another preferred embodiment of the invention.

Another subject matter of the present invention is a detergent, cleaning agent or care agent containing particles according to the invention as described above and/or as obtainable by a method according to the invention.

Particles according to the invention may be incorporated into a solid detergent or cleaning agent with no problem. A preferred solid detergent or cleaning agent can contain 0.1 to 20 wt %, preferably 1 to 10 wt % of the particles according to the invention, which can be simply mixed together, for example.

Another subject matter of the present invention lies in the use of the particles according to the invention, as described above, or the detergent, cleaning agent or care agent according to the invention, as described above, in textile laundry and/or treatment, preferably in an automatic washing machine.

Detergents or cleaning agents according to the invention can also contain surfactant(s) in addition to the particles according to the invention. Anionic, nonionic, zwitterionic and/or amphoteric surfactants may be used. From the standpoint of application technology, mixtures of anionic and nonionic surfactants are preferred. Total surfactant content of a detergent is preferably greater than 5 wt %, or better yet greater than 10 wt %, but advantageously less than 40 wt % and especially preferably less than 35 wt %, based on total detergent.

Preferably, alkoxylated, advantageously ethoxylated, in particular primary alcohols with preferably 8 to 18 carbon atoms and an average of 1 to 12 mol ethylene oxide (EO) per mol alcohol may be used as the nonionic surfactants, wherein the alcohol radical may be linear or preferably having a methyl branch in position 2 and/or may contain linear and methyl-branched radicals in the mixture such as those usually present in oxo alcohol radicals. In particular, alcohol ethoxylates with linear radicals from alcohols of native origin with 12 to 18 carbon atoms, for example, from coconut, palm, tallow fat or oleyl alcohol, and an average of 2 to 8 EO per mol alcohol are preferred. Preferred ethoxylated alcohols include C₁₂₋₁₄ alcohols with 3 EO, 4 EO or 7 EO, C₉₋₁₁ alcohol with 7 EO, C₁₃₋₁₅ alcohols with 3 EO, 5 EO, 7 EO or 8 EO, C₁₂₋₁₈ alcohols with 3 EO, 5 EO or 7 EO and mixtures thereof such as mixtures of C₁₂₋₁₄ alcohol with 3 EO and C₁₂₋₁₈ alcohol with 7 EO. The stated degrees of ethoxylation represent statistical averages, which may be an integral number or a fraction for a specific product. Preferred alcohol ethoxylates have a narrow homolog distribution (narrow-range ethoxylates, NRE). In addition to these nonionic surfactants, fatty alcohols with more than 12 EO may also be used. Examples include tallow fatty alcohol with 14 EO, 25 EO, 30 EO or 40 EO. According to the invention, nonionic surfactants containing EO and PO groups together in the molecule may also be used. Block copolymers with EO-PO block units and/or PO-EO block units may be used here, but EO-PO-EO copolymers and/or PO-EO-PO copolymers may also be used. Mixed alkoxylated nonionic surfactants may of course also be used, in which EO and PO units are not distributed by blocks but instead are randomly distributed. Such products can be obtained by simultaneous action of ethylene oxide and propylene oxide on fatty alcohols. Furthermore, alkyl glycosides of the general formula RO(G)ₓ, in which R denotes a primary linear or methyl-branched aliphatic radical, in particular with the methyl branching in position 2, having 8 to 22, preferably 12 to 18 carbon atoms, and G is the symbol standing for a glycose unit with 5 or 6 carbon atoms, preferably glucose, may also be used as additional nonionic surfactants. The degree of oligomerization x, which indicates the distribution of monoglycosides and oligoglycosides, is any number between 1 and 10; x is preferably 1.2 to 1.4. Alkyl glycosides are known as mild surfactants. Another class of nonionic surfactants which may preferably be used either as the sole nonionic surfactant or in combination with other nonionic surfactants include alkoxylated, preferably ethoxylated or ethoxylated and propoxylated fatty acid alkyl esters, preferably with one to four carbon atoms in the alkyl chain, in particular fatty acid methyl esters. Nonionic surfactants of the amine oxide type, for example, N-coconut alkyl-N,N-dimethylamine oxide and N-tallow-alkyl-N,N-dihydroxyethylamine oxide and the fatty acid alkanolamides may also be suitable. The amount of these nonionic surfactants is preferably no more than that of the ethoxylated fatty alcohols, in particular no more than half therefore. The optional nonionic surfactant content in the detergents or cleaning agents is preferably >0.1 wt %, for example, 5 to 30 wt %, preferably 7 to 20 wt % and in particular 9 to 15 wt %, each based on the total detergent or cleaning agent. In another embodiment, the detergent or cleaning agent does not contain any nonionic surfactants or only small amounts, e.g., <0.5 wt %.

Useful anionic surfactants include those of the sulfonate and sulfate types. Preferred surfactants of the sulfonate type include C₉₋₁₃-alkylbenzenesulfonates, olefinsulfonates, meaning, mixtures of alkenesulfonates and hydroxyalkanesulfonates as well as disulfonates such as those obtained from C₁₂₋₁₈ monoolefins with terminal or internal double bonds by sulfonation with gaseous sulfur trioxide and subsequent alkaline or acidic hydrolysis of the sulfonation products. Also suitable are alkanesulfonates obtained from C₁₂₋₁₈ alkanes, for example, by sulfochlorination or sulfoxidation with subsequent hydrolysis and/or neutralization. Likewise, the esters of α-sulfo fatty acids (ester sulfonates), for example, the α-sulfonated methyl esters of hydrogenated coconut, palm kernel or tallow fatty acids are also suitable. Other suitable anionic surfactants include sulfated fatty acid glycerol esters. Fatty acid glycerol esters are understood to be the mono-, di- and triesters as well as mixtures thereof, such as those obtained in synthesis by esterification of a monoglycerol with 1 to 3 mol fatty acid or in transesterification of triglycerides with 0.3 to 2 mol glycerol. Preferred sulfated fatty acid glycerol esters include the sulfation products of saturated fatty acids with 6 to 22 carbon atoms, for example, caproic acid, caprylic acid, capric acid, myristic acid, lauric acid, palmitic acid, stearic acid or behenic acid. Preferred alk(en)yl sulfates are the alkali salts, particularly the sodium salts of sulfuric acid hemiesters of C₁₂-C₁₈ fatty alcohols, for example, those from coconut fatty alcohol, tallow fatty alcohol, lauryl, myristyl, cetyl or stearyl alcohol or the C₁₀-C₂₀ oxo alcohols and the hemiesters of secondary alcohols of these chain lengths. Also preferred are the alk(en)yl sulfates of the aforementioned chain length containing a synthetic linear alkyl radical synthesized petrochemically and having a degradation behavior similar to that of the adequate compounds based on the raw materials of fat chemistry. Of interest from the standpoint of washing technology are the C₁₂-C₁₆ alkyl sulfates and C₁₂-C₁₅ alkyl sulfates as well as the C₁₄-C₁₅ alkyl sulfates. 2,3-Alkyl sulfates, commercially available under the brand name DAN® from the Shell Oil Company, are also preferred anionic surfactants. Preferred anionic surfactants are soaps in particular. Saturated and unsaturated fatty acid soaps such as the salts of lauric acid, myristic acid, palmitic acid, stearic acid, (hydrogenated) erucaic acid and behenic acid as well as in particular soap mixtures derived from natural fatty acids, for example, coconut, palm kernel, olive oil or tallow fatty acids are preferred in particular. The anionic surfactants including the soaps may be present in the form of their sodium, potassium or ammonium salts as well as soluble salts of organic bases such as mono-, di- or triethanolamine. The anionic surfactants are preferably present in the form of their sodium or potassium salts, in particular in the form of the sodium salts. The optional anionic surfactant content of preferred detergents or cleaning agents preferably amounts to >0.1 wt %, for example, 2 to 30 wt %, preferably 4 to 25 wt % and in particular 5 to 22 wt %, each based on the total detergent or cleaning agent.

In addition to the particles according to the invention and the optional surfactants, the detergents or cleaning agents may also contain other ingredients which further improve the esthetic properties and/or technical application-related properties of the detergents or cleaning agents. Within the scope of the present invention, preferred detergents or cleaning agents may additionally contain one or more substances from the group of builders, bleaches, bleach activators, enzymes, perfumes, perfume carriers, fluorescent agents, dyes, foam inhibitors, silicone oils, antiredeposition agents, optical brighteners, graying inhibitors, shrinkage preventers, wrinkle control agents, dye transfer inhibitors, antimicrobial active ingredients, germicides, fungicides, antioxidants, preservatives, corrosion inhibitors, antistatics, bittering agents, ironing aids, phobicizing agents and impregnating agents, swelling agents and nonslip agents, neutral filler salts and UV absorbers.

Builders that may be present in the detergents or cleaning agents include in particular silicates, aluminum silicates (zeolites in particular), carbonates, salts of organic di- and polycarboxylic acids as well as mixtures of these substances. In another preferred embodiment, the detergent or cleaning agent does not contain any zeolite. Organic builders which may be present in the detergents or cleaning agents include polycarboxylate polymers, such as polyacrylates and acrylic acid/maleic acid copolymers, polyaspartates and monomeric polycarboxylates such as citrates, gluconates, succinates or malonates which are preferably used as sodium salts. The total amount of the builders optionally used, comprising, for example, zeolite, polycarboxylate, sodium citrate, is preferably 1-70 wt %. Appropriate lower limits may be 10, 15, 20 or 30 wt %, for example. Appropriate upper limits may be 40, 55 or 60 wt %, for example.

Of the compounds which supply H₂O₂ in water and serve as bleaching agents, sodium perborate tetrahydrate and sodium perborate monohydrate are especially important. Other bleaching agents that may be used include, for example, sodium percarbonate, peroxypyrophosphates, citrate perhydrates and per acid salts, which supply H₂O₂, or per acids such as perbenzoates, peroxophthalates, diperazelaic acid, phthalimino per acid or diperdodecanoic diacid. The total amount of bleaching agents optionally included may be, for example, 5-25 wt % or preferably also 10-20 wt %, if the presence of bleaching agents is desired.

The detergents or cleaning agents may contain enzymes in encapsulated form and/or directly in the detergent or cleaning agent. Enzymes that may be used include in particular those from the classes of hydrolases such as proteases, esterases, lipases and/or lipolytic enzymes, amylases, cellulases and/or other glycosyl hydrolases, hemicellulase, cutinases, β-glucanases, oxidases, peroxidases, perhydrolases and/or laccases and mixtures of the aforementioned enzymes. The enzymes may be adsorbed onto carrier substances to protect them from premature decomposition. The amount of enzymes or enzyme granules directly in the detergent or cleaning agent may be, for example, approximately 0.01 to 5 wt %, preferably 0.12 to approximately 2.5 wt %.

In one embodiment, the detergent or cleaning agent may optionally contain one or more perfumes in an amount of up to about 10 wt %, preferably 0.5 to 7 wt %, in particular 1 to 3 wt %. The amount of perfume used also depends on the type of detergent or cleaning agent. However, it is preferred that the perfume be introduced into the detergent or cleaning agent at least partially through the particles according to the invention. However, it is also possible for the detergent or cleaning agent to contain perfume which is not introduced into the detergent or cleaning agent via the particles according to the invention.

Soil-release polymers may usually be used in amounts from 0% to, for example, 5 wt %, based on finished detergent or cleaning agent. Optical brighteners may usually be used in amounts from 0% to 0.3 wt %, based on finished detergent or cleaning agent.

The amount of optional dye transfer inhibitor, based on total amount of detergent or cleaning agent, is preferably 0.01 to 2 wt %, especially preferably 0.05 to 1 wt % and more preferably from 0.1 to 0.5 wt %.

Heavy-metal-chelating substances may also be used to prevent heavy-metal-catalyzed decomposition of certain detergent ingredients. Suitable heavy-metal-chelating agents include alkali salts of ethylenediaminetetraacetic acid (EDTA) or nitrilotriacetic acid (NTA), as well as alkali metal salts of anionic polyelectrolytes, such as polymaleates and polysulfonates.

A preferred class of chelating agents include phosphonates, present in preferred detergents or cleaning agents in amounts of 0.01 to 2.5 wt %, preferably 0.02 to 2 wt %, and in particular from 0.03 to 1.5 wt %. These preferred compounds include organophosphates such as 1-hydroxyethane-1,1-diphosphonic acid (HEDP), aminotri(methylenephosphonic acid) (ATMP), diethylenetriamine-penta(methylenephosphonic acid) (DTPMP and/or DETPMP), as well as 2-phosphonobutane-1,2,4-tricarboxylic acid (PBS-AM), for example, most of which may be used in the form of their ammonium salts or alkali metal salts.

In addition, neutral filler salts such as sodium sulfate or sodium carbonate may also be present in the solid detergents or cleaning agents.

Detergents or cleaning agents according to the invention may also be used for cleaning and conditioning textile fabrics.

To produce detergents or cleaning agents according to the invention, the detergent or cleaning agent is first produced by known methods which may include, for example, drying steps, mixing steps, compaction steps, shaping steps and/or the subsequent addition of heat-sensitive ingredients (“post addition”). Next, the resulting product is mixed with particles according to the invention. To produce molded bodies of detergent or cleaning agent, additional compaction steps and/or shaping steps may be performed following the mixing step.

### EXAMPLE

Table 2 shows particles E1 to E3 according to the invention. Numerical data in Table 2 is given in wt %.

TABLE 2

| | E1 | E2 | E3 |
|---|---|---|---|
| Sucrose crystals (0.5 to 3 mm) | 70.998 | 78.9989 | 77.998 |
| Bentonite | 4 | 4 | 4 |
| Silica | 4 | 3 | 4 |
| Perfume | 3 | 4 | 2 |
| Polydimethylsiloxane | 7 | — | — |
| Polyquaternium 7 | — | 1 | — |
| Polyquaternium 10 | — | — | 2 |
| Perfume microcapsules | 5 | 4 | 4 |
| PEG 6000 | 6 | 5 | 6 |
| Dye | 0.002 | 0.002 | 0.002 |

We claim:

1. Particle suitable for use in laundry-detergent, cleaning-agent or care products, comprising:
a water-soluble or water-dispersible carrier, and
microcapsules comprising one or more active ingredients.

2. Particle according to claim 1, wherein the water-soluble or water-dispersible carrier further comprises one or more materials chosen from inorganic alkali metal salts, organic alkali metal salts, inorganic alkaline earth metal salts, organic alkaline earth metal salts, organic acids, carbohydrates, silicates, urea or mixtures thereof.

3. Particle according to claim 1, wherein the water-soluble or water-dispersible carrier further comprises a carbohydrate chosen from dextrose, fructose, galactose, isoglucose, glucose, sucrose, raffinose or mixtures thereof.

4. Particle according to claim 1, wherein the carrier is in the form of crystals.

5. Particle according to claim 1, wherein the active ingredient microcapsules comprise a liquid active ingredient suitable for laundry, cleaning, care and/or finishing purposes.

6. Particle according to claim 5, wherein the liquid active ingredient is chosen from
(a) fragrances,
(b) textile-care ingredients, and/or
(c) skin-care ingredients.

7. Particle according to claim 1, wherein the active ingredient is at least perfume present in an amount of from 0.01 to 30 wt %, wt % based on total particle.

8. Particle according to claim 7, wherein the amount of perfume contained in the microcapsules is 0.1 to 20 wt %, wt % based on total particle.

9. Particle according to claim 1 further comprising a thermoplastic polymer present in an amount of 0.01-25 wt %, wt % based on total particle.

10. Particle according to claim 1 further comprising one or more water-binding substances present in an amount of 0 to 20 wt %, wt % being based on the total particle, wherein the one or more water-binding substances are chosen from zeolite, silica, textile-softening clay, starch and/or derivatives thereof and/or cellulose and/or derivatives thereof.

11. Particle according to claims 1, wherein the water-soluble or water-dispersible carrier further comprises a coating comprising a mixture of thermoplastic polymer and microcapsules.

12. Particle according to claim 1, wherein the microcapsules are water-insoluble microcapsules.

13. Particle according to claim 1, wherein the water-soluble or water-dispersible carrier has a particle size of from about 0.1 to about 30 mm.

14. Method for producing particles according to claim 1, comprising
(a) producing a mixture of microcapsules and thermoplastic polymer in the form of a melt containing microcapsules, and
(b) mixing the mixture of the melt from step (a) with water-soluble or water-dispersible carrier material.

15. Method according to claim 13, wherein the microcapsules in step (a) are in the form of an aqueous slurry mixed into the melt together with water-binding substances.

16. Detergents, cleaning agents or care agents comprising particles according to claim 1.
